(19) United States
(12) Patent Application Publication
(10) Pub. No.: US 2023/0067194 A1
(43) Pub. Date: Mar. 2, 2023

(54) METHOD FOR TREATMENT OF ACQUIRED COGNITIVE DEFICITS

(71) Applicant: CORNELL UNIVERSITY, lthaca, NY (US)

(72) Inventors: Jackie L. GOTTSHALL, Gaithersburg, MD (US); Nicholas SCHIFF, New York, NY (US); Diany Paola CALDERON, New York, NY (US)

(73) Assignee: CORNELL UNIVERSITY, Ithaca, NY (US)

(21) Appl. No.: 17/794,109

(22) PCT Filed: Jan. 27, 2021
(86) PCT No.: PCT/US21/15155

§ 371 Date: Jul. 20, 2022

Related U.S. Application Data
(60) Provisional application No. 62/966,287, filed on Jan. 27, 2020.

(51) Int. Cl. A61B 5/00

(52) U.S. Cl. CPC A61B 5/68; A61B 5/7235

(57) ABSTRACT

The present disclosure is directed to methods for improving a wakeful function in a subject suffering from an acquired cognitive deficiency by administering to the subject an effective amount of an upregulator of GABA_{B} signaling, thereby improving the wakeful function in the subject. Another aspect of the disclosure us directed to methods for improving a wakeful function in a subject suffering from an acquired cognitive deficiency by administering to the subject an effective amount of an upregulator of GABA_{B} signaling at night, in combination with administering an anterior-forebrain stimulating therapy when the subject is awake, thereby improving the wakeful function in the subject.

### CROSS REFERENCE TO RELATED APPLICATION

This application claims the benefit of priority from U.S. Provisional Application No. 62/966,287, filed Jan. 27, 2020, the entire contents of which are incorporated herein by reference.

### BACKGROUND

Loss of organized sleep electrophysiology is a characteristic finding following severe brain injury. The return of structured elements of sleep architecture has been associated with positive prognosis across injury etiologies, suggesting a role for sleep dynamics as biomarkers of wakeful neuronal circuit function. Increasing evidence suggests that many patients with disorders of consciousness experience neuronal re-organization and recovery of large-scale brain function over prolonged time periods. The presence of sleep architecture, particularly sleep spindles and slow wave sleep (SWS), has been found to correlate with prognosis following injury and therefore may be an important dimension for understanding and tracking functional improvements. Adams et al. (Clin Neurophysiol (2016) 127:3086-3092) first reported on the electroencephalogram (EEG) sleep characteristics of this patient before and during approximately five years of daytime central thalamic deep brain stimulation (CT-DBS), which was initiated for the promotion of arousal regulation. Initial findings by Adams et al. (Clin Neurophysiol (2016) 127:3086-3092) showed an increase in sleep spindle frequency and SWS duration following the onset of CT-DBS treatment. An irregular intrusion of alpha activity during SWS was also reported prior to CT-DBS, which seemed to attenuate during CT-DBS treatment. Importantly, these results implicated daytime brain activation as modulator of sleep architecture in the severely injured brain.

At present, there is a lack of therapeutic options for patients who experience continued cognitive deficits either beyond the sub-acute phase following brain injury or in conjunction with chronic disease or psychiatric disturbance. This therapeutic gap remains despite growing evidence that (1) cognitive deficits persist in a significant portion of patients after even mild traumatic brain injury, and (2) neuronal circuits can retain an enduring capacity for reorganization and reconstitution of function across the spectrum of physiological insults ranging from mild to severe structural brain injuries and over decades of life. The significance of developing a therapeutic intervention for individuals suffering from acquired cognitive deficits lies in the loss of autonomy and functional independence that dramatically reduces their quality of life, as well as the substantial economic burden imposed by the chronic healthcare needs of these individuals. Thus, there is a significant need for treatment methods aimed at restoring neuronal circuits responsible for cognitive function in patients with acquired cognitive deficits.

### SUMMARY OF THE DISCLOSURE

An aspect of this disclosure is directed to a method for improving a wakeful function in a subject suffering from an acquired cognitive deficiency, comprising administering to the subject an effective amount of an upregulator of gamma-aminobutyric acid B (GABA_{B}) signaling, thereby improving the wakeful function in the subject.

In some embodiments, the administering step is performed at night.

In some embodiments, the upregulator of GABA_{B} signaling is selected from the group consisting of baclofen, tiagabine, lesogaberan (AZD3355), GS-39783, zolpidem, midazolam, and sodium oxybate.

In some embodiments, the upregulator of GABA_{B} signaling is represented by the chemical formula:

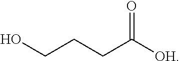

In some embodiments, the upregulator of GABA_{B} signaling is sodium oxybate with the following chemical formula:

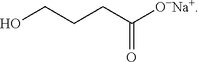

In some embodiments, the upregulator of GABA_{B} signaling is represented by the chemical formula:

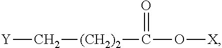

wherein X is H, a pharmaceutically acceptable cation or (C₁-C₄)alkyl, and Y is OH, (C₁-C₄)alkoxy, (C₁-C₄) alkanoyloxy or benzoyloxy. In some embodiments, Y is OH or (C₁-C₄) alkanoyloxy. In some embodiments, X is Na⁺.

In some embodiments, the upregulator of GABA_{B} signaling comprises electrical stimulation of corticothalamic/thalamocortical afferents in the subject.

In some embodiments, the acquired cognitive deficiency results from an insult selected from the group consisting of stroke, toxicological agents, anoxia, ischemia, nutritional deficiencies, developmental diseases, infectious diseases, neoplastic diseases, and degenerative diseases.

In some embodiments, the acquired cognitive deficiency results from a traumatic brain injury.

In some embodiments, the wakeful function is selected from the group consisting of perceptual awareness, memory, intellect, learning ability, logic ability, attention and executive function.

In some embodiments, the method also improves one or more features of sleep selected from the group consisting of depth of slow wave modulation, frequency of spindles, REM sleep, and sleep architecture.

Another aspect of the disclosure is directed to a method for improving a wakeful function in a subject suffering from an acquired cognitive deficiency, comprising administering to the subject an effective amount of an upregulator of GABA_{B} signaling at night, in combination with administering an anterior-forebrain stimulating therapy when the subject is awake, thereby improving the wakeful function in the subject.

In some embodiments, the anterior-forebrain stimulating therapy comprises administering a small molecule compound selected from the group consisting of amantadine hydrochloride, methylphenidate, donazepil, noradrenaline, and ketamine.

In some embodiments, the anterior-forebrain stimulating therapy is selected from the group consisting of deep brain stimulation, transcranial direct current stimulation, optogenetic stimulation, and focused ultrasound.

In some embodiments, the upregulator of GABA_{B} signaling is selected from the group consisting of baclofen, tiagabine, lesogaberan (AZD3355), GS-39783, zolpidem, midazolam, and sodium oxybate.

In some embodiments, the upregulator of GABA_{B} signaling is represented by the chemical formula:

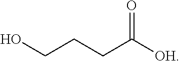

In some embodiments, the upregulator of GABA_{B} signaling is sodium oxybate represented by the chemical formula:

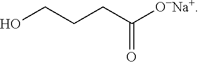

In some embodiments, the upregulator of GABA_{B} signaling is represented by the chemical formula

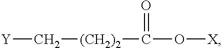

wherein X is H, a pharmaceutically acceptable cation or (C₁-C₄)alkyl, and Y is OH, (C₁-C₄)alkoxy, (C₁-C₄) alkanoyloxy or benzoyloxy. In some embodiments, Y is OH or (C₁-C₄) alkanoyloxy. In some embodiments, X is Na⁺.

In some embodiments, the upregulator of GABA_{B} signaling comprises electrical stimulation of corticothalamic/thalamocortical afferents in the subject.

In some embodiments, the acquired cognitive deficiency results from an insult selected from the group consisting of stroke, toxicological agents, anoxia, ischemia, nutritional deficiencies, developmental diseases, infectious diseases, neoplastic diseases, and degenerative diseases.

In some embodiments, the acquired cognitive deficiency results from a traumatic brain injury.

In some embodiments, the wakeful function is selected from the group consisting of perceptual awareness, memory, intellect, learning ability, logic ability, attention and executive function.

In some embodiments, the method also improves one or more features of sleep selected from the group consisting of depth of slow wave modulation, frequency of spindles, REM sleep, and sleep architecture.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B. Representative EEG and summary timeline. (A) Representative segments of patient subject EEG tracings during non-REM sleep show the presence of stage two (left panel) and SWS (middle, right panels) epochs. A mixed frequency signature consistent with alpha delta sleep was also observed, (middle panel), classified here as a subset of SWS for consistency with combined “SWS-like” states reported in Adams et al., (Clin Neurophysiol (2016) 127:3086-3092). (B) The patient subject was studied at five time points over the course of 8.5 years, consisting of one time point before, three during, and one after CT-DBS treatment. Qualitative summary of individual EEG states are shown for each time point.

FIGS. 2A-2B. Behavioral examination scores. (A) Coma Recovery Scale-Revised (CRS-R) total scores at each time point. Each data point represents a single CRS-R administration. *, p<0.05. (B) Corresponding CRS-R subscale scores display slight variations in composition of total CRS-R scores between time points. Each data point represents a single subscale administration. Grey rectangles indicate maximum subscale score range. Data points from pre CT-DBS are shown in grey, active CT-DBS in blue, and post CT-DBS in black.

FIGS. 3A-3C. EEG power spectra from T1, T4 and T5, corresponding to pre-, active, and post-CT-DBS conditions. (A) Power spectra calculated from resting awake EEG. Arrows indicate changes in peak frequency in the alpha range. * indicates emergence of a “wicket rhythm” (˜8-13 Hz) over the left motor cortex. FC6 and C4 inset arrows denote increases in peak alpha frequency at time point 4 (B-C) Power spectra calculated from non-REM stage two (B) and SWS (C). Spectral tracings from time point 1 (T1) are represented by grey lines, time point 4 (T4) by blue lines, and time point 5 (T5) by black lines.

FIGS. 4A-4B. Stage two sleep spindle dynamics Power spectra were calculated for representative stage two sleep EEG segments and values normalized for comparison across time points. (A) Power in the 9-16 Hz spindle range, plotted according to channel and time point. (B) Peak spectral frequency in the 9-16 Hz spindle range. If no spectral peak was present, spindles were considered absent and no frequency value was recorded. Dotted lines represent left hemisphere channels and solid lines represent right hemisphere channels. Individual channels are plotted by color. Grey shading indicates active CT-DBS time points.

FIGS. 5A-5B. Slow wave sleep dynamics. Power spectra were calculated for representative SWS segments (including alpha delta sleep) and values normalized for comparison across time points. (A) SWS power in the 0.5-4 Hz delta frequency range, plotted according to channel and time point. (B) SWS power in the 8-14 Hz alpha frequency range, plotted according to channel and time point. Dotted lines represent left hemisphere channels and solid lines represent right hemisphere channels. Individual channels are plotted by color. Grey color indicates active CT-DBS time points.

FIGS. 6A-6B. Proposed mechanism linking CT-DBS and the alleviation of alpha-delta sleep. An integrative hypothesis linking the observed effects of CT-DBS in our patient subject with anterior forebrain mesocircuit function and sleep dynamics (A) Anterior forebrain mesocircuit dysfunction in disorders of consciousness. In severe brain injury, widespread deafferentation results in functional downregulation of the prefrontal cortex (PFC) via functional disfacilitation and structural deafferentation of central thalamic neuronal populations. Under these conditions, medium spiny neurons of the striatum fail to reach firing threshold, resulting in released inhibition of the globus pallidus interna (GPi). Excess firing of the GPi is proposed to result in additional inhibition of central thalamic components, preventing sufficient thalamocortical output and tilting the excitatory/inhibitory balance needed for the consistent maintenance of consciousness. Such reduced activation of the PFC during wake may result in insufficient accumulation of homeostatic sleep pressure and correspondingly under-activated prefrontal GABAergic networks during sleep. Failure to inhibit ventral limbic structures such as the basal forebrain would result in excess activation of thalamocortical cells and the intrusion of alpha oscillations during SWS. (B) Restoration of mesocircuit function and alpha-delta sleep alleviation during CT-DBS. CT-DBS drives thalamocortical output, resulting in restored wakeful excitation of the PFC. Increased PFC engagement during wakefulness produces an accumulation of homeostatic sleep pressure, facilitating the inhibition of ventral limbic structures during sleep via upregulation of SWS-producing GABAergic interneurons. Without excess thalamic activation by ventral structures during SWS, depolarization necessary for alpha-producing high threshold thalamocortical bursting is not achieved and alpha-delta sleep does not occur.

FIGS. 7A-7B. Effects of sodium oxybate injection on the sleep-wake cycle of SBI mice. The top panels display the delta power during the dark (blue; daytime) and light (yellow; sleep time) within 24 hours in (A) Non-REM, or (B) Wake mice. The pink vertical lines represent the time in which animals received an intraperitoneal injection of sodium oxybate (SO; 400 mg/Kg) diluted in saline. The bottom panels show the LFP power spectra obtained from sleep periods and wake periods with and without sodium oxybate. In (A), the dark blue line represents NREM sleep without sodium oxybate administration and the red line represents NREM sleep with sodium oxybate administration. In (B), the dark blue line represents Wake without sodium oxybate administration during prior sleep and the red line represents Wake with sodium oxybate administration during prior sleep. Application of sodium oxybate during the night results in elevation of delta (1-5 Hz) LFP power and increases during the subsequent wakeful period in beta (15-30 Hz) and gamma (>30 Hz) LFP. Importantly, consistent with our theory because of the short half-life of sodium oxybate these changes in wakeful LFP power reflect alteration of cortical neuronal properties linked to the increased delta during sleep induced by sodium oxybate. Similarly, the elevation of delta power does not persist during the wakeful period.

### DETAILED DESCRIPTION

### Subject

In some embodiments, the methods of the instant disclosure are directed to a subject who suffers from an acquired cognitive deficiency.

As used herein, “acquired cognitive deficiency” refers to subnormal functioning or a suboptimal functioning in one or more wakeful functions such as perceptual awareness, memory, intellect, learning and logic ability, or attention and executive function (working memory) in a particular individual comparative to other individuals within the same general age population due to an insult to the individual's brain or in comparison with the individuals prior baseline function. The insult to the individual's brain may include, but is not limited to brain injuries, including those produced, at least in part, by stroke, head trauma (e.g., blast injury, blunt head trauma, or missile penetration), toxicological agents (e.g., carbon monoxide, arsenic, or thallium), anoxia (e.g., reduced oxygen levels in the blood), ischemia (e.g., reduced blood flow), nutritional deficiencies, developmental diseases, infectious diseases, neoplastic diseases, degenerative diseases, complications thereof, or other structural lesions.

These brain injuries frequently manifest themselves in one or more deficits of wakeful functions including, but not limited to, attention, intention, working memory, logic ability and/or awareness. As used herein, attention refers to the cognitive function that provides the capacities for selection of internal or external stimuli and thoughts, supports the preparation of intended behaviors (e.g., speeds perceptual judgments and reaction times), and supports the maintenance of sustained cognition or motor behaviors (e.g., the focusing of attention). Intention, as used herein, refers to the mechanism of response failures (i.e., lack of behavioral interaction) which is not due to a perceptual loss (i.e., intention is the cognitive drive linking sensory-motor integration to behavior). Intention deficits include failure to move a body part despite intact motor pathways, awareness, and sensory processing as demonstrated by neurophysiological and neuropsychological evaluation. Another example of a patient's intention deficit is a failure to initiate action of any kind despite evidence of awareness or action produced by stimulation. Loss of intention is a disorder of cognitive function, as defined herein, and is a major division of the neuropsychological disorder of neglect, which may be present in many patients with cognitive loss following brain injury. Working memory, as used herein, refers to the fast memory process required for on-line storage and retrieval of information, including processes of holding incoming information in short-term memory before it can be converted into long-term memory and processes which support the retrieval of established long-term (episodic) memories. Deficits in awareness relate to impaired perceptual awareness, as described above. Clinical signs of these brain injuries also include profound hemi-spatial neglect, disorders of motor intention, disorders of impaired awareness of behavioral control, or apathy and cognitive slowing.

In a specific embodiment, the acquired cognitive deficiency results from a traumatic brain injury, i.e., the subject suffers from a traumatic brain injury. A “traumatic brain injury” refers to a brain injury caused by a physical blow/mechanical impact/over-pressurization impulse/blast injury, e.g., to the head or body, that results in damage to the brain tissue, vascular system, or surrounding tissue or bone (e.g., the skull).

A subject's wakeful functions, including attention, intention, working memory, and/or awareness function, can be evaluated using standard tests. Standard tests evaluate different types of basic cognitive functions and are used to initially screen a patient for a pattern of deficits. More specific tests can be employed and individualized to a patient's neuropsychological profile. In practice, the choice of particular neuropsychological test batteries depends on the experience of the tester and the normative data available for the test. This changes as new studies are done and as new testing materials are tried out and compared. For example, suitable comprehensive tests include the Mental Status Exam (“MSE”) (set forth, for example, in Strub et al., The Mental Status Exam in Neurology, 3rd ed., Philadelphia: Davis (1993), which is hereby incorporated by reference) as well as broad neuropsychological test batteries, like the Halstead-Reitan Neuropsychological Test Battery (which encompasses memory, attention, intention, and perception/awareness). In order to delineate more narrowly specific deficits of working memory, attention, perception, etc., more individualized tests can be chosen. For example, a ‘Shipley-Hartford scale’ test may be employed to assess cognitive slowing (intelligence); a ‘Bender-Gestalt’ test can be used to assess spatial relations and constructions; Aphasia screening tests, such as the Boston Diagnostic Aphasia Examination or the Western Aphasia Battery, can detect language dysfunction; and Trials A/B or Memory Assessment Scales (“MAS”) test can be used to assess working memory. Further details with regard to these and other tests for assessing a patient's attention, intention, working memory, and/or awareness function can be found in, for example, Berg, “Screening Tests in Clinical Neuropsychology,” Chapter 10, pp. 331-363, in Horton et al., eds., The Neuropsychology Handbook, Vol. 1, Foundations and Assessment, 2nd ed., New York: Springer Publishing Company (1997), which is hereby incorporated by reference.

In some embodiments, the subject is an animal. The term “animal” includes mammals, for example, human, horse, camel, dog, pig, cow, and sheep. In a specific embodiment, the subject is a human.

### Upregulators of GABA_{B} Signaling

An “upregulator of GABA_{B} signaling” is a modulator that activates the GABA_{B} receptor (GABA_{B}R). Without being bound to a particular theory, it is believed that activation of GABA_{B}R stimulates the opening of K⁺ channels (e.g., G protein-coupled inwardly-rectifying potassium channels (GIRKs)), which brings the neuron closer to the equilibrium potential of K⁺. This reduces the frequency of action potentials which reduces neurotransmitter release.

In some embodiments, the upregulator of GABA_{B} signaling is a small molecule compound that activates GABA_{B}R. The term “small molecule compound” herein refers to small organic chemical compound, generally having a molecular weight of less than 2000 daltons, 1500 daltons, 1000 daltons, 800 daltons, or 600 daltons.

In some embodiments, the upregulator of GABA_{B} signaling selected from the group consisting of baclofen, tiagabine, lesogaberan (AZD3355), GS-39783, zolpidem, midazolam, and sodium oxybate.

In some embodiments, the upregulator of GABA_{B} signaling is represented by the formula:

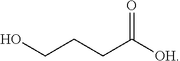

In some embodiments, the upregulator of GABA_{B} signaling is sodium oxybate with the following chemical formula:

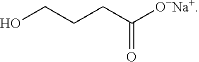

In some embodiments, the upregulator of GABA_{B} signaling is represented by the formula:

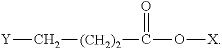

wherein X is H, a pharmaceutically acceptable cation or (C₁-C₄)alkyl, and Y is OH, (C₁-C₄)alkoxy, (C₁-C₄) alkanoyloxy or benzoyloxy. In some embodiments, Y is OH or (C₁-C₄) alkanoyloxy. In some embodiments, X is Na⁺.

In some embodiments, the upregulator of GABA_{B} signaling is a small molecule compound selected from the small molecule compounds disclosed in U.S. Pat. No. 5,990,162, which is incorporated herein by reference.

In some embodiments, the upregulator of GABA_{B} signaling is baclofen represented by the chemical formula:

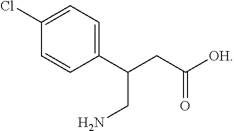 [SMILES: NCC(CC(=O)O)c1ccc(Cl)cc1]

In some embodiments, the upregulator of GABA_{B} signaling is tiagabine represented by the chemical formula:

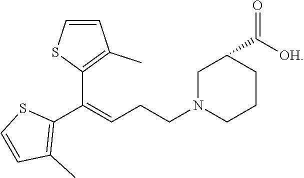 [SMILES: Cc1ccsc1C(=CCCN1CCC[C@@H](C(=O)O)C1)c1sccc1C]

In some embodiments, the upregulator of GABA_{B} signaling is lesogaberan (AZD3355) represented by the chemical formula:

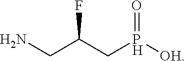

In some embodiments, the upregulator of GABA_{B} signaling is GS-39783 represented by the chemical formula:

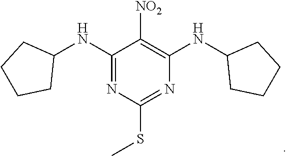 [SMILES: CSc1nc(NC2CCCC2)c([N+](=O)[O-])c(NC2CCCC2)n1]

In some embodiments, the upregulator of GABA_{B} signaling is zolpidem represented by the chemical formula:

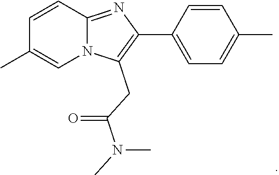

In some embodiments, the upregulator of GABA_{B} signaling is midazolam represented by the chemical formula:

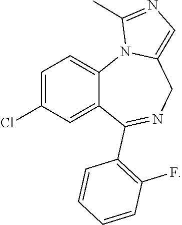 [SMILES: Cc1ncc2n1-c1ccc(Cl)cc1C(c1ccccc1F)=NC2]

In some embodiments, the upregulator of GABA_{B} signaling is electrical stimulation of corticothalamic afferents. In some embodiments, electrical stimulation of corticothalamic afferents is achieved as described herein below.

### Anterior-Forebrain Stimulating Therapies

As used herein, the phrase “anterior-forebrain stimulating therapy” refers to therapies that activate the anterior-forebrain part of the brain.

In some embodiments, the anterior-forebrain stimulating therapy is achieved by electrical stimulation of the anterior-forebrain region of a subject.

In some embodiments, electrical stimulation is achieved by electrodes. A variety of electrodes can be employed for delivering the stimulation. For example, suitable electrodes include the deep brain stimulation electrodes used in Katayama, “Characterization and Modification of Brain Activity with Deep Brain Stimulation in Patients in a Persistent Vegetative State: Pain-Related Late Positive Component of Cerebral Evoked Potential,” Pace, 14:116-121 (1991), which is hereby incorporated by reference, and the Medtronic DBS 3280 (available from Medtronic, Minneapolis, Minn.), which has a flexible TEFLON-SILASTIC™ coated, platinum iridium electrodes with 4 contacts, 4 mm tips, 2 mm, mean tip separation, and an impedance of 5-7 kΩ within the brain, described in Velasco et al., “Electrocortical and Behavioral Responses Produced By Acute Electrical Stimulation of the Human Centromedian Thalamic Nucleus,” Electroencephalography and Clinical Neurophysiolocy, 102:461-471 (1997) (“Velasco”), which is hereby incorporated by reference. In some embodiments, the electrode is an implantable multipolar electrode with either an implantable pulse generator that can be under patient control or a radiofrequency controlled device operated by an external transmitter. In some embodiments, the multipolar electrode contacts should allow for adjustment of frequency (or “rate”), amplitude, and pulse width within at least the following respective ranges: about 2-200 Hz, about 0.1-10 Volts, and about 50-500 microseconds. As used in this disclosure, the term “about” refers to ±10% of a given value. In some embodiments, the multipolar electrode contacts allow for adjustment in a broader range than those recited above, particularly toward higher intensities. Such electrodes include a Medtronic 3387 electrode (available from Medtronic, Minneapolis, Minn.) and are described, for example, in Benabid et al., “Chronic Electrical Stimulation of the Ventralis Intermedius Nucleus of the Thalamus As a Treatment of Movement Disorders,” J. Neurosurgery, 84:203-214 (1996), which is hereby incorporated by reference. In some situations, it may be desirable to use an electrode capable of delivering pharmacological agents to the patient's anterior-forebrain.

The electrode can be contacted with the patient's anterior-forebrain by the methods conventionally employed for embedding or emplacing electrodes for deep brain electrical stimulation in other thalamic nuclei. Such methods are described in Tasker et al., “The Role of the Thalamus in Functional Neurosurgery,” Neurosurgery Clinics of North America, 6(1):73-104 (1995) (“Tasker”), which is hereby incorporated by reference. Briefly, the multi-polar electrode or electrodes are introduced via burr holes in the skull. The burr holes are placed based on the particular region of the anterior-forebrain to be contacted. In some embodiments, prior to the introduction of the implantable multi-polar electrode(s), a detailed mapping with microelectrode and microstimulation following standard methods is carried out as described in Tasker, which is hereby incorporated by reference. Briefly, for each subdivision of the anterior-forebrain, a preferred trajectory of approach optimizing the safety of entry point and maximal number of identifiable physiological landmarks in the responses of cell groups encountered along the trajectory into the desired region or regions of the anterior-forebrain can be identified by one skilled in the art. This can be done, for example, by following the methods and catalogued physiological responses of different human thalamic cell groups described in Tasker, which is hereby incorporated by reference. Initial mapping of the path for the stimulating electrode(s) can, therefore, be carried out via a combination of detailed single-unit recording of receptive field (“RF”) properties of the cells encountered along the trajectory, projective fields (“PF”) mapped by microstimulation of the same cell groups, and comparison with known RF and PF responses in the human thalamus. Similarly, evoked potentials can be recorded and, for the anterior-forebrain, have several characteristic signatures identifiable from scalp surface recording as discussed in Velasco and Tasker, which are hereby incorporated by reference. For this mapping, microstimulation, using tungsten microelectrodes with impedances of roughly 1.5 megaohms, every 1 mm at threshold of up to 100 microamperes with short trains of 300 Hz pulses of 0.2 millisecond pulse width are employed as described in Tasker, which is hereby incorporated by reference. Typically, an on-line data base of RF and PF information along the trajectory and stereotactic coordinates derived, for example, from Schaltenbrand et al., Introduction to Stereotaxis with an Atlas of the Human Brain, Stuttgart: Thieme (1977), which is hereby incorporated by reference, or by computed mapping techniques, such as those described in Tasker et al., “Computer Mapping of Brainstem Sensory Centres in Man,” J. Neurosurg., 44:458-464 (1976), which is hereby incorporated by reference, can be used, either with or without a magnetic resonance imaging (“MRI”) based stereotactic apparatus. To carry out the above methods a patient would typically remain conscious with application of local anesthesia or mild sedation. However, in cases where a patient is not sufficiently cooperative to remain conscious during the procedure, the above-described approach can be modified to allow the operation to be completed under general anesthesia.

The electrical stimulation can be continuous, intermittent, or periodic. The range of stimulation frequencies and intensity of stimulation will depend on several factors: impedance of the electrode once in the brain, excitation properties of cells which may differ within subdivisions of the anterior-forebrain, the type of induced physiologic responses sought for a particular patient, and inter-individual variation. While higher frequency ranges are thought to be preferred, lower frequencies will also be employed. Suitable stimulation frequencies range from about 1 Hz to 1 kHz; from about 10 Hz to about 500 Hz; and from about 50 Hz to about 250 Hz. In some embodiments, the stimulation frequency ranges between 1 Hz and 1000 Hz, between 10 Hz and 500 Hz, between 100 Hz and 250 Hz, between 200 Hz and 350 Hz, between 300 Hz and 450 Hz, between 400 Hz and 550 Hz, between 500 Hz and 650 Hz, between 600 Hz and 750 Hz, between 700 Hz and 850 Hz, between 800 Hz and 950 Hz, or between 900 Hz and 1000 Hz. In some embodiments, the frequency of the electrical stimulation is about 1 Hz, about 10 Hz, about 50 Hz, about 100 Hz, about 150 Hz, about 200 Hz, about 250 Hz, about 300 Hz, about 350 Hz, about 400 Hz, about 450 Hz, about 500 Hz, about 550 Hz, about 600 Hz, about 650 Hz, about 700 Hz, about 750 Hz, about 800 Hz, about 850 Hz, about 900 Hz, or about 1000 Hz.

Typically, the electrode is connected to an insulated conductor which leads to an external connector plug which is removably connected to a mating plug which is, in turn, connected to a voltage control and pulse generator. The pulse generator produces a selected pulse train, and the voltage control provides a selected current amplitude or voltage to the waves of the pulse train. In some embodiments, both the voltage control and pulse generator are under control of a computer microprocessor. The signal pulse generator should be capable of generating voltage wave trains of any desired form (sine, square wave, spike, rectangular, triangular, ramp, etc.) in a selectable voltage amplitude in the range from about 0.1 volts to about 10 volts and at selectable frequencies as set forth above. In practice, the pulse train and voltage amplitudes employed will be selected on a trial and error basis by evaluating a patient's response to various types and amplitudes of electrical stimulation over a time course of from about 1 to about 12 months. For example, after implanting the electrode in the patient's anterior-forebrain, stimulation with a voltage within the range of from about 0.1 to about 10 volts or higher (e.g., about 0.1V, about 0.5V, about 1V, about 1.5V, about 2V, about 2.5V, about 3V, about 3.5V, about 4V, about 4.5V, about 5V, about 5.5V, about 6V, about 6.5V, about 7V, about 7.5V, about 8V, about 8.5V, about 9V, about 9.5V, about 10V), a rate within the range of from about 50 to about 250 Hz, and a pulse width within the range of from about 50 to about 500 microseconds is applied for from about 8 to about 12 hours a day. In some embodiments, the pulse width is about 20 microseconds, about 30 microseconds, about 40 microseconds, about 50 microseconds, about 60 microseconds, about 70 microseconds, about 80 microseconds, about 90 microseconds, about 100 microseconds, about 150 microseconds, about 200 microseconds, about 250 microseconds, about 300 microseconds, about 350 microseconds, about 400 microseconds, about 450 microseconds, about 500 microseconds. During and after the implantation of the electrode, the parameters of the stimulation (voltage, pulse width, and frequency) are adjusted to optimize the patient's interactive behavior. The stimulation parameters can be further optimized by monitoring the patient clinically, anatomically, physiologically, or metabolically to assess his or her response to the stimulation. For example, the patient can be physiologically examined using electroencephalogram (“EEG”), magnetoencephalogram (“MEG”), or functional magnetic resonance imaging (“fMRI”). Metabolic evaluation can be carried out, for example, using quantitative fluorodeoxy-glucose positron emission tomography (“FDG-PET”). In addition, correlation of induced changes in surface electrical brain activity (as measured, for example, by EEG or MEG) can be correlated with improved function and increased resting metabolism of a damaged brain regions as identified by PUG-PET. Careful evaluation of these different indices of brain function in conjunction with standard neurological and neuropsychological tests can thus be used to optimize the beneficial effect of the stimulation method of the present disclosure.

In some embodiments, the electrical stimulation is a deep brain stimulation method taught in U.S. Pat. Nos. 5,938,688 and 6,539,263, both of which are incorporated by reference in their entirety.

In some embodiments, the anterior-forebrain stimulating therapy is achieved by a treatment selected from the group consisting of deep brain stimulation, transcranial direct current stimulation, optogenetic stimulation, and focused ultrasound.

In some embodiments, the anterior-forebrain stimulating therapy is achieved by administering a small molecule compound selected from the group consisting of amantadine hydrochloride, methylphenidate, donazepil, noradrenaline, and ketamine.

In some embodiments, the small molecule compound used for anterior-forebrain stimulating therapy is amantadine hydrochloride represented by the chemical formula:

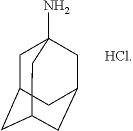 [SMILES: NC12CC3CC(CC(C3)C1)C2]

In some embodiments, the small molecule compound used for anterior-forebrain stimulating therapy is methylphenidate represented by the chemical formula:

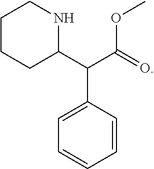 [SMILES: COC(=O)C(c1ccccc1)C1CCCCN1]

In some embodiments, the small molecule compound used for anterior-forebrain stimulating therapy is donazepil represented by the chemical formula:

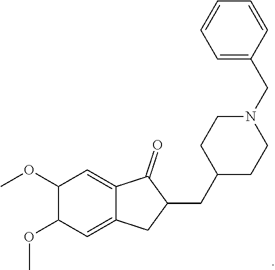 [SMILES: COC1C=C2CC(CC3CCN(Cc4ccccc4)CC3)C(=O)C2=CC1OC]

In some embodiments, the small molecule compound used for anterior-forebrain stimulating therapy is noradrenaline (norepinephrine) represented by the chemical formula:

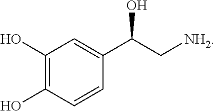 [SMILES: NC[C@H](O)c1ccc(O)c(O)c1]

In some embodiments, the small molecule compound used for anterior-forebrain stimulating therapy is ketamine represented by the chemical formula:

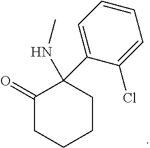 [SMILES: CNC1(c2ccccc2Cl)CCCCC1=O]

Methods for Improving a Wakeful Function in a Subject Suffering from an Acquired Cognitive Deficiency

This disclosure is premised, in part, by a recognition uniquely provided by the present inventors that GABAergic upregulation has a bidirectional carry-over effect between sleep and wakeful states. Upregulation of frontocortical GABAergic circuits during wakefulness resulted in the improvement of sleep architecture, and upregulation of GABAergic circuits during sleep resulted in the improvement or recovery of wakeful frontocortical function. Thus, activation of GABAergic circuits in a state-dependent manner exerts a 24 hour cyclical influence over organized neuronal function. Therefore, according to one aspect of this disclosure, in a subject suffering from acquired cognitive deficiency, wakeful cognition can be improved by upregulating GABA_{B} signaling during sleep (e.g., by administering a modulator before and/or during sleep that upregulates GABA_{B} signaling). According to another aspect, in a subject suffering from acquired cognitive deficiency, wakeful cognition can be improved by upregulating GABA_{B} signaling during sleep (e.g., by administering a modulator before and/or during sleep that upregulates GABA_{B} signaling), in combination with upregulation of GABAergic signaling during wakefulness. While improving wakeful functions in a subject having acquired cognitive deficiency, the present treatment approach, i.e., upregulation GABA_{B} signaling during sleep alone or in combination with upregulation of GABAergic signaling during wakefulness, also improves sleep physiology in the subject including improvement of normal sleep features (e.g., increasing the depth of slow wave modulation, increasing frequency of spindles, restoration of REM, and normalization of sleep architecture and circadian rhythmicity), as well as remediation of abnormal sleep features (e.g., eliminating alpha-delta sleep pattern).

### Administration of an Upregulator of GABA_{B} Signaling

An aspect of this disclosure is directed to a method for improving a wakeful function in a subject suffering from an acquired cognitive deficiency, comprising administering to the subject an effective amount of an upregulator of GABA_{B} signaling as described herein, thereby improving the wakeful function in the subject. In some embodiments, the wakeful function is selected from the group consisting of perceptual awareness, memory, intellect, learning ability, logic ability, attention and executive function.

In some embodiments, the administering step is performed at night. In some embodiments, the administering step is performed when the subject is asleep. In some embodiments, the administering step is performed about two hours, one and a half hours, one hour, half an hour, 15 minutes, ten minutes, or 5 minutes before subject falls asleep. In some embodiments, an upregulator of GABA_{B} signaling is administered intermittently every hour, every two hours, every three hours, every six hours or every twelve hours, as appropriate, while the subject is asleep or between bouts of sleep.

In some embodiments, an upregulator of GABA_{B} signaling is administered to a subject in need thereof every day. In some embodiments, an upregulator of GABA_{B} signaling is administered intermittently every 2, 3, 4, 5, or 6 days, or every week, every 2, 3, or 4 weeks, as appropriate.

In some embodiments, the upregulator of GABA_{B} signaling is a small molecule compound selected from the group consisting of baclofen, tiagabine, lesogaberan (AZD3355), GS-39783, zolpidem, midazolam, and sodium oxybate.

In some embodiments, an upregulator of GABA_{B} signaling is administered to a subject at a dose between 0.2 mg/kg and 300 mg/kg, depending on the particular compound used and the route of administration chosen. In other embodiments, an upregulator of GABA_{B} signaling is administered at a dose about 0.2 mg/kg, 0.5 mg/kg, 8 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 150 mg/kg, 175 mg/kg, 200 mg/kg, 250 mg/kg, 275 mg/kg or 300 mg/kg.

In some embodiments, an upregulator of GABA_{B} signaling disclosed herein can be administered by any of the conventional routes of administration, including but not limited to oral, nasal, sublingual, buccal, or parenteral (including e.g., subcutaneous, intramuscular, transcutaneous, intradermal, intraperitoneal, intraocular, and intravenous) route. In some embodiments, an upregulator of GABA_{B} signaling is administered in combination with a pharmaceutically acceptable carrier. In some embodiments, an upregulator of GABA_{B} signaling is administered by inhalation. In some embodiments, an upregulator of GABA_{B} signaling is administered by means of a transdermal patch.

In some embodiments, the upregulator of GABA_{B} signaling is electrical stimulation of corticothalamic afferents.

In some embodiments, corticothalamic afferents are electrically stimulated in a subject in need thereof every day. In some embodiments, corticothalamic afferents are electrically stimulated in a subject in need thereof intermittently every 2, 3, 4, 5, or 6 days, or every week, every 2, 3, or 4 weeks, as appropriate. In some embodiments, corticothalamic/thalamocortical afferents are electrically stimulated in the subject intermittently every hour, every two hours, every three hours, every six hours or every twelve hours, as appropriate, while the subject is asleep.

In some embodiments, the acquired cognitive deficiency results from an insult selected from the group consisting of stroke, toxicological agents, anoxia, ischemia, nutritional deficiencies, developmental diseases, infectious diseases, neoplastic diseases, and degenerative diseases

In some embodiments, the acquired cognitive deficiency results from a traumatic brain injury. In some embodiments, the subject does not suffer from daytime sleepiness or sleep fragmentation. In some embodiments, the subject has normal REM sleep before the therapy.

In some embodiments, the method also improves one or more features of sleep selected from the group consisting of depth of slow wave modulation, frequency of spindles, REM sleep, circadian rhythmicity, and sleep architecture. In some embodiments, the method improves the depth of slow wave modulation during sleep. In some embodiments, the method increases the frequency of spindles (also called “sleep spindles”, “sigma bands” or “sigma waves”). In some embodiments, the method causes reemergence of REM sleep. In some embodiments, the method improves sleep architecture, meaning that the subject has longer uninterrupted sleep and longer deep sleep during the night.

Another aspect of the disclosure is directed to use of an upregulator of GABA_{B} signaling in production of a medicament for improving a wakeful function in a subject suffering from an acquired cognitive deficiency.

Yet another aspect of the disclosure is directed to an upregulator of GABA_{B} signaling for use in improving a wakeful function in a subject suffering from an acquired cognitive deficiency.

### Combination Therapy

Another aspect of this disclosure is directed to a combination therapy of an upregulator of GABA_{B} signaling and an anterior-forebrain stimulating therapy for improving a wakeful function in a subject suffering from an acquired cognitive deficiency. In some embodiments, the method comprises administering to the subject an effective amount of an upregulator of GABA_{B} signaling (as described herein) at night, in combination with administering an anterior-forebrain stimulating therapy when the subject is awake, thereby improving the wakeful function in the subject.

In some embodiments, an upregulator of GABA_{B} signaling administered at night. In some embodiments, an upregulator of GABA_{B} signaling administered when the subject is asleep. In some embodiments, an upregulator of GABA_{B} signaling is administered about two hours, one and a half hours, one hour, half an hour, 15 minutes, ten minutes, or 5 minutes before subject falls asleep.

In some embodiments, an anterior-forebrain stimulating therapy is administered during the day. In some embodiments, an anterior-forebrain stimulating therapy is administered when the subject is awake. In some embodiments, an anterior-forebrain stimulating therapy is administered about three hours, four hours, five hours, six hours, seven hours, or ten hours before subject falls asleep.

In some embodiments, the upregulator of GABA_{B} signaling is a small molecule compound selected from the group consisting of baclofen, tiagabine, lesogaberan (AZD3355), GS-39783, zolpidem, midazolam, and sodium oxybate.

In some embodiments, an upregulator of GABA_{B} signaling is administered to a subject at a dose between 0.2 mg/kg and 300 mg/kg, depending on the particular compound used and the route of administration chosen. In other embodiments, an upregulator of GABA_{B} signaling is administered at a dose about 0.2 mg/kg, 0.5 mg/kg, 8 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 150 mg/kg, 175 mg/kg, 200 mg/kg, 250 mg/kg, 275 mg/kg or 300 mg/kg.

In some embodiments, an upregulator of GABA_{B} signaling disclosed herein can be administered by any of the conventional routes of administration, including but not limited to oral, nasal, sublingual, buccal, or parenteral (including e.g., subcutaneous, intramuscular, transcutaneous, intradermal, intraperitoneal, intraocular, and intravenous) route. In some embodiments, an upregulator of GABA_{B} signaling is administered in combination with a pharmaceutically acceptable carrier. In some embodiments, an upregulator of GABA_{B} signaling is administered by inhalation. In some embodiments, an upregulator of GABA_{B} signaling is administered by means of a transdermal patch.

In some embodiments, the upregulator of GABA_{B} signaling is electrical stimulation of corticothalamic/thalamocortical afferents.

In some embodiments, the anterior-forebrain stimulating therapy is achieved by a direct stimulation method selected from the group consisting of deep brain stimulation, transcranial direct current stimulation, optogenetic stimulation, and focused ultrasound.

In some embodiments, the anterior-forebrain stimulating therapy comprises administering a small molecule compound selected from the group consisting of amantadine hydrochloride, methylphenidate, donazepil, noradrenaline, and ketamine, as described herein.

In some embodiments, a small molecule compound for the anterior-forebrain stimulating therapy is administered to a subject at a dose between 0.2 mg/kg and 300 mg/kg, depending on the particular compound used and the route of administration chosen. In other embodiments, a small molecule compound for the anterior-forebrain stimulating therapy is administered at a dose about 0.2 mg/kg, 0.5 mg/kg, 8 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 150 mg/kg, 175 mg/kg, 200 mg/kg, 250 mg/kg, 275 mg/kg or 300 mg/kg.

In some embodiments, a small molecule compound for the anterior-forebrain stimulating therapy disclosed herein can be administered by any of the conventional routes of administration, including but not limited to oral, nasal, or parenteral (including e.g., subcutaneous, intramuscular, transcutaneous, intradermal, intraperitoneal, intraocular, and intravenous) route. In some embodiments, a small molecule compound for the anterior-forebrain stimulating therapy is administered in combination with a pharmaceutically acceptable carrier. In some embodiments, a small molecule compound for the anterior-forebrain stimulating therapy is administered by inhalation. In some embodiments, a small molecule compound for the anterior-forebrain stimulating therapy is administered by means of a transdermal patch.

In some embodiments, an upregulator of GABA_{B} signaling and an anterior-forebrain stimulating therapy are administered to a subject in need thereof every day. In some embodiments, an upregulator of GABA_{B} signaling and an anterior-forebrain stimulating therapy are administered intermittently every 2, 3, 4, 5, or 6 days, or every week, every 2, 3, or 4 weeks, as appropriate. In each administration schedule, the upregulator of GABA_{B} signaling is administered at night at most one hour before sleep or when the subject is asleep, and an anterior-forebrain stimulating therapy administered when the subject is awake.

In some embodiments, the subject suffers from a traumatic brain injury. In some embodiments, the subject does not suffer from daytime sleepiness or sleep fragmentation. In some embodiments, the subject has normal REM sleep before the therapy.

In some embodiments, the method also improves one or more features of sleep selected from the group consisting of depth of slow wave modulation, frequency of spindles, REM sleep, circadian rhythmicity, and sleep architecture. In some embodiments, the method improves the depth of slow wave modulation during sleep. In some embodiments, the method increases the frequency of spindles (also called “sleep spindles”, “sigma bands” or “sigma waves”). In some embodiments, the method causes reemergence of REM sleep. In some embodiments, the method improves sleep architecture, meaning that the subject has longer uninterrupted sleep and longer deep sleep during the night.

Another aspect of the disclosure is directed to use of an upregulator of GABA_{B} signaling and an anterior-forebrain stimulating therapy in production of a medicament for improving a wakeful function in a subject suffering from an acquired cognitive deficiency.

Yet another aspect of the disclosure is directed to an upregulator of GABA_{B} signaling and an anterior-forebrain stimulating therapy for use in improving a wakeful function in a subject suffering from an acquired cognitive deficiency.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The specific examples listed below are only illustrative and by no means limiting.

### EXAMPLES

### Example 1

### I. Materials and Methods

### Patient History

Patient subject is a 48-year-old man who suffered a severe brain injury as the result of a motor vehicle accident at the age of 17. The injury pattern is characterized by a small left thalamic hemorrhage, as well as diffuse axonal injury with extensive atrophy of the left hemisphere. Behavioral presentation has remained consistent with a diagnosis of minimally conscious state since the time of injury.

### Data Collection & Timeline

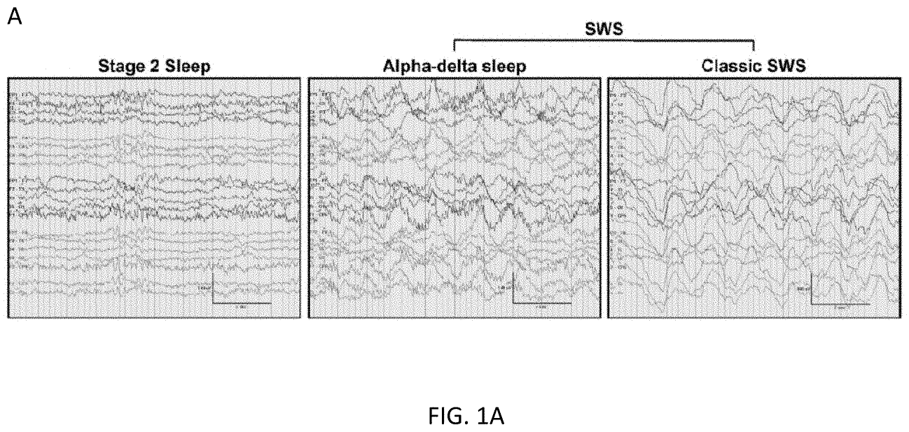

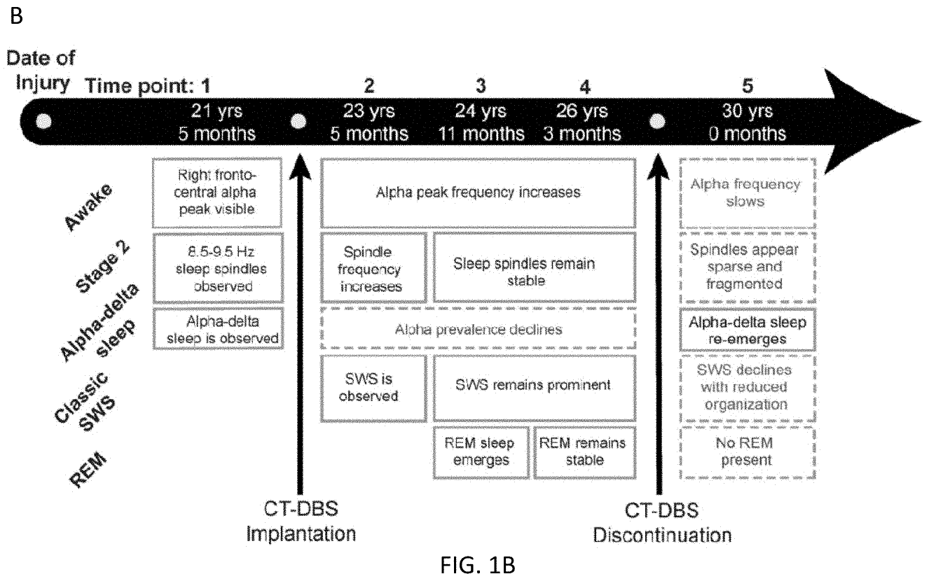

The patient subject was studied longitudinally at five time points (T1-T5) over the course of 8.5 years (FIG. 1B). Each study consisted of a 24-72 hour inpatient admission (T1 at New York Presbyterian Hospital, New York, USA; T2-T5 at Rockefeller University Hospital, New York, USA) under IRB approvals from Weill Cornell Medicine and Rockefeller University. Written informed consent was obtained from the patient's surrogate for study participation, data collection and publication. During each inpatient study, behavioral responsiveness was quantified according to the Coma Recovery Scale-Revised (CRS-R) (17) at least once per day. Overnight video-EEG was collected with collodion-pasted electrodes (30 electrodes at T1; 37 electrodes at T2-T5) placed according to the international 10-20 system. Signals were recorded using the Natus XLTEK system (San Carlos, Calif.) at impedances≤5 kΩ Time point one occurred at 21 years 5 months post injury. The patient subject subsequently underwent surgery for the implantation of bilateral central thalamic deep brain stimulation electrodes (clinical trial methods are described briefly below, as well as in detail in Schiff et al., 2007 (Nature (2007) 448:600-603)). Study time points two through four were concurrent with daytime central thalamic deep brain stimulation (CT-DBS) (T2: 23 years 5 months, T3: 24 years 11 months, and T4: 26 years 3 months post injury, respectively). Time point five occurred approximately one year following CT-DBS discontinuation secondary to battery depletion (T5: 30 years 0 months post injury).

Implantation of deep brain stimulation electrode leads was aided by microelectrode recordings from the sensory relay nucleus of the thalamus on both the right and left hemispheres. Using electrophysiological localization of the sensory nucleus as known, the lateral wing of the central lateral nucleus was targeted using the Morel atlas (Morel A. et al, J Comp Neurol., (1997) 387:588-630) by dead reckoning. Following implantation, CT-DBS was administered on a blocked 12 hour ON/OFF cycle (ON: 6 AM-6 PM/OFF: 6 PM-6 AM) over the course of approximately 7½ years. During this time, a wide range of electrode contact geometries, stimulation intensities, and frequencies of stimulation were employed. Briefly, following extensive titration testing of stimulation frequency and intensity, an optimal geometry was identified for each electrode contact based on apparent arousal effects and limitations of visible side effects. The left electrode was stimulated in bipolar mode with the lowest contact chosen as cathode and highest contact chosen as anode; the right electrode was stimulated in monopolar mode with two cathodes, placed at the lowest two contacts. During a 6-month crossover phase, stimulation at these contacts occurred for 12 hours each day using a 90-microsecond pulse width, 130 Hz stimulation frequency, and 4V intensity for each electrode. Following the crossover phase, a range of varying frequencies and intensities were used including a one-year period of stimulation at each of 175 Hz and 100 Hz. For the majority of the 7½ years of CT-DBS exposure stimulation occurred at 100 Hz with other parameters held constant.

### Data Analysis

### EEG Processing

For estimates of wakeful brain dynamics, periods of resting eyes open awake states were identified by video record and corresponding EEG was manually cleaned for the removal of eye blink and movement artifacts.

Sleep analyses included nighttime sleep EEG collected between the hours of 8 PM and 6 AM, manually cleaned for movement artifacts and verified eyes-closed according to synchronous video record. Standard sleep scoring criteria were used to classify segments as stage two or slow wave sleep (Tatum WO., Neurodiagn J., (2016) 56:285-293). Briefly, the patient subject was considered to be in stage two sleep if the EEG record displayed k-complexes and/or 9-16 Hz spindle-like formations across frontocentral channels. Slow wave sleep was classified by large polymorphic delta (<4 Hz) waves present over at least 20% of a 30-second epoch. The observation of an additional and constant 8-14 Hz oscillation overriding classic SWS characteristics was considered alpha-delta sleep. To maintain consistency with the previous report by Adams et al. (Clin Neurophysiol (2016) 127:3086-3092), alpha-delta sleep was scored under the categorization of SWS.

### Power Spectral Estimation

Raw EEG was segmented into 30-35 representative epochs for awake, stage two sleep, and SWS states respectively. Multitaper power spectral estimates were calculated separately for each state (Thomson DJ., Proc IEEE (1982) 70:1055-1096) with implementation of Hjorth Laplacian montaging from the MATLAB chronux toolbox (Bokil H. et al., J Neurosci Methods, (2010) 192:146-151). After spectral calculation, six frontocentral channels (F3, F4, FCS, FC6, C3, C4) were used for longitudinal comparison to maintain consistency with Adams et al. (Clin Neurophysiol (2016) 127:3086-3092).

For longitudinal comparisons of spectral peak sizes during stage two and SWS stages, calculated spectra were normalized according to methods outlined in Gottselig et al., (Brain (2002) 125:373-383). Briefly, a power law function was fit to each spectrum in the 5-6 Hz and 17-18 Hz frequency ranges for stage two sleep, or 4-6 Hz and 23-24 Hz frequency ranges for SWS. Frequency ranges for normalization were chosen based on optimal fitting of the power law function to the underlying shape of the power spectrum across channels, excluding frequency bins of interest to avoid flattening of relevant spectral features. Absolute power of the fitted spectrum was subtracted from the calculated spectrum and resulting values were integrated across frequency bins of interest. By subtracting the best-fit underlying spectral shape, arbitrary differences in background power bias between visits were removed, allowing for estimation of magnitude change in relevant spectral features.

Dominant spindle frequency was determined from normalized stage two power spectra using a handcrafted manual click program to determine the center frequency of the largest spectral peak in the 9-16 Hz spindle range. Briefly, the spindle peak for each channel was visually identified from the normalized power spectrum and a quadratic polynomial was fit to the identified peak to determine the local power maxima and corresponding dominant spindle frequency. If no spindle peak was present no value was recorded.

### Statistics

Analyzed variables were CRS-R total score, stage two sleep spindle power (9-16 Hz) and peak spindle frequency, SWS delta power (0.5-4 Hz), and SWS alpha power (8-14 Hz). Time periods for comparison were grouped into three conditions reflecting initial pre-stimulation baseline, the active period of CT-DBS, and the post-withdrawal of stimulation phase (Pre:T1/Active:T2-T4/Post:T5). An analysis of variance (ANOVA) was performed for each variable to identify changes across CT-DBS condition. For stage two and SWS variables, ANOVA factors included CT-DBS condition and hemisphere. To identify any changes within the active CT-DBS condition, a separate ANOVA was conducted for T2-T4 within each variable. Post-hoc comparisons were conducted using Tukey's HSD at a significance level of p<0.05.

### II. Results

In a continuing study of one minimally conscious state patient studied over the course of approximately 8½ years, the inventors sought to investigate whether changes in daytime brain activation induced by central thalamic deep brain stimulation (CT-DBS) influenced sleep electrophysiology. In this patient subject, the inventors previously reported significant improvements in sleep electrophysiology during 5½ years of CT-DBS treatment, including increased sleep spindle frequency and SWS delta power. The inventors herein present novel findings that many of these improvements in sleep electrophysiology regress following CT-DBS discontinuation; these regressions in sleep features correlate with a significant decrease in behavioral responsiveness. The inventors also observe the re-emergence of alpha-delta sleep, which had been previously suppressed by daytime CT-DBS in this patient subject. Importantly, CT-DBS was only active during the daytime and has been proposed to mediate recovery of consciousness by driving synaptic activity across frontostriatal systems through the enhancement of thalamocortical output. Accordingly, the improvement of sleep dynamics during daytime CT-DBS and their subsequent regression following CT-DBS discontinuation implicates wakeful synaptic activity as a robust modulator of sleep electrophysiology. The inventors interpret these findings in the context of the “synaptic homeostasis hypothesis”, whereby we propose that daytime upregulation of thalamocortical output in the severely injured brain may facilitate organized frontocortical circuit activation and yield net synaptic potentiation during wakefulness, providing a homeostatic drive that reconstitutes sleep dynamics over time. Furthermore, the inventors consider common large-scale network dynamics across several neuropsychiatric disorders in which alpha-delta sleep has been documented, allowing us to formulate a novel mechanistic framework for alpha-delta sleep generation. The inventors conclude that the bi-directional modulation of sleep electrophysiology by daytime thalamocortical activity in the severely injured brain: 1) emphasizes the cyclical carry-over effects of state-dependent circuit activation on large-scale brain dynamics, and 2) further implicates sleep electrophysiology as a sensitive indicator of wakeful brain activation and covert functional recovery in the severely injured brain.

Here, the inventors report on distinct changes observed approximately one year after the discontinuation of CT-DBS treatment. The inventors find ongoing plasticity in multiple physiological aspects of sleep, providing important insight into the network-level dynamics that can be induced by daytime arousal regulation in the severely injured brain. Most notably, in the present study the inventors identify a regression of the previously noted improvements in sleep dynamics seen over course of CT-DBS treatment. A return of the atypical SWS alpha intrusion initially reported by Adams et al. as “mixed state” is evaluated here in the context of the previously documented phenomenon “alpha-delta sleep” (Adams et al., Clin Neurophysiol (2016) 127:3086-3092). The inventors discuss the findings in the context of neuronal circuit mechanisms that may organize the improvement of sleep dynamics during daytime CT-DBS in the severely injured brain, as well as those that may underlie functional regression with the withdrawal of CT-DBS treatment. Finally, the inventors explore the role of alpha-delta sleep across pathophysiologies of neuropsychiatric disorders and propose a mechanistic explanation for alpha-delta sleep generation.

### Visual EEG Features

FIG. 1A provides a qualitative summary of changes in EEG architecture over the course of study. Most notable was the observation at T1 of an additional sleep signature consisting of high voltage, low frequency (<2 Hz) activity exhibiting an overriding mid-frequency (8-14 Hz) component (FIG. 1A, middle panel). This signature closely resembles alpha-delta sleep, characterized by Hauri and Hawkins (Electroencephalogr Clin Neurophysiol (1973), 34:233-237) as “a mixture of 5-20% delta waves (>75 uV, 0.5-2 c/sec) combined with relatively large amplitude, alpha-like rhythms (7-10 c/sec)”. Alpha-delta sleep was prominent before CT-DBS treatment (T1), waned during active CT-DBS (T2-4), and re-emerged following discontinuation of CT-DBS (T5). Inversely, changes in healthy sleep architecture during CT-DBS treatment included the normalization of stage two sleep spindles, SWS, and awake alpha rhythms, as well as the emergence of REM sleep. Each of these healthy features demonstrated qualitative decline following CT-DBS discontinuation (FIG. 1B).

### Behavioral Examination

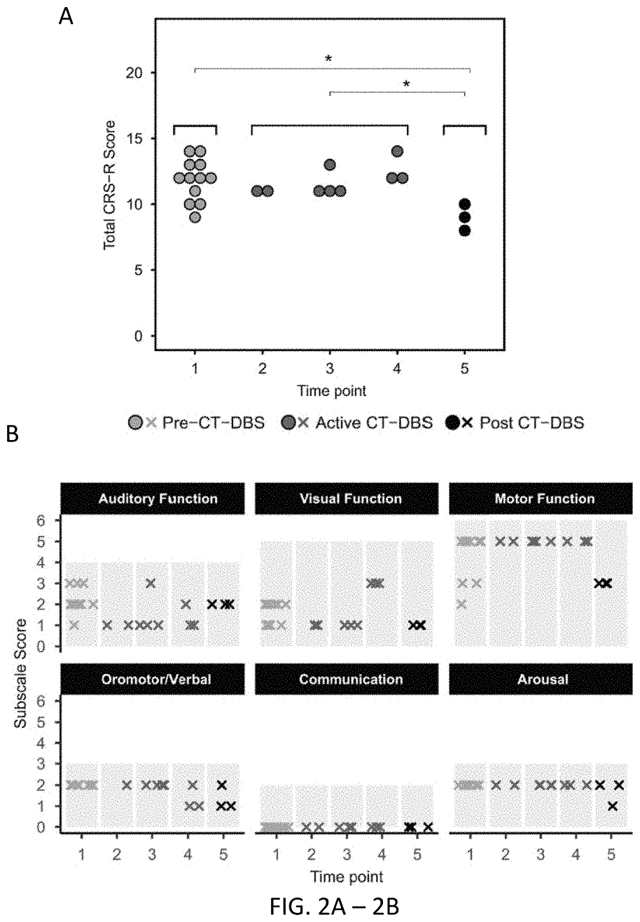

The CRS-R was administered at least once daily during each time point. A one-way ANOVA showed a significant effect of CT-DBS condition (pre, active, post) [F(2, 21)=5.55, p=0.0116], such that total CRS-R scores were significantly lower after CT-DBS cessation (M=9.0, SD=1.0) than either before CT-DBS (M=11.8, SD=1.6, p=0.011) or during active CT-DBS (M=11.8, SD=1.1, p=0.016) (FIG. 2A). Although this reduction in CRS-R score was statistically significant, the patient subject remained within the diagnostic classification of minimally conscious state throughout the course of study. There was no change in CRS-R scores between active CT-DBS time points.

CRS-R subscale scores were also compared for a detailed view of composite CRS-R score changes. Analysis of variance was not performed due to the categorical nature of subscale classifications. Subscale scores varied slightly across time points, with the exception of the communication subscale, for which the patient subject received a score of 0 at each examination (FIG. 2B). Altogether, although CT-DBS did not produce an increase in CRS-R scores, the withdrawal of CT-DBS correlated with a significant reduction in responsiveness at T5.

### Power Spectra During Wake, Stage 2, and SWS

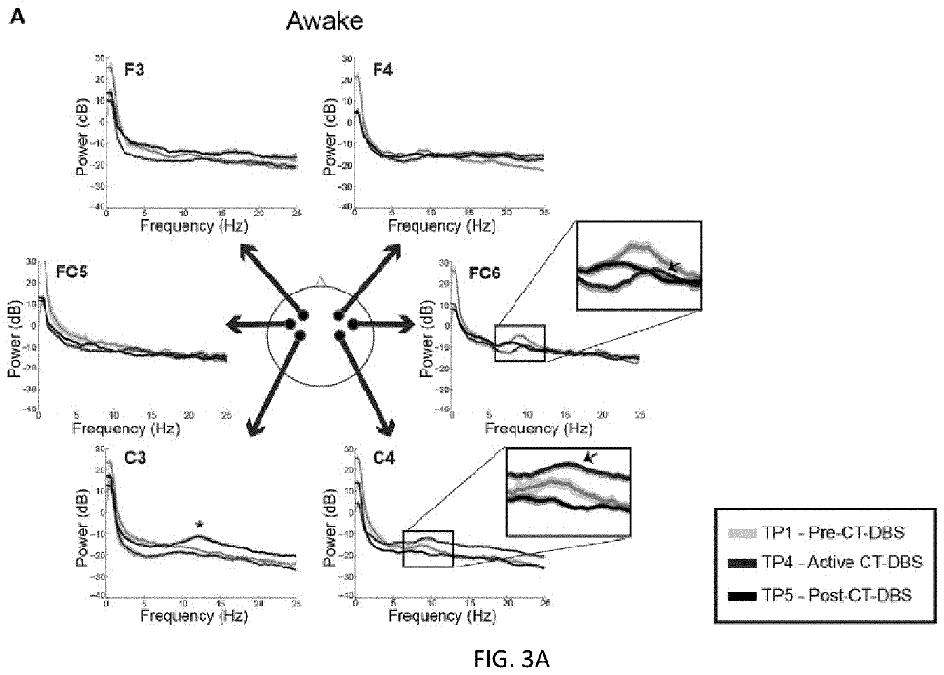

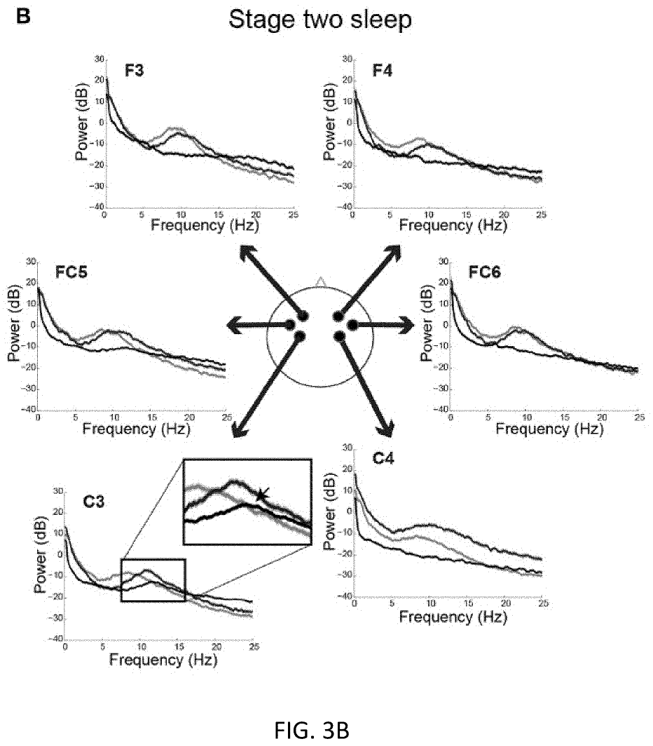

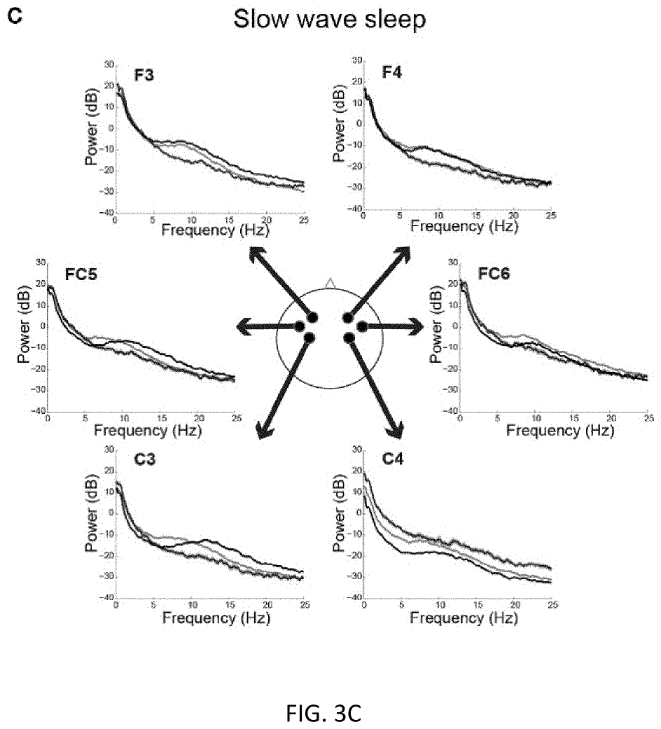

Power spectra from T1, T4, and T5 were overlaid for a qualitative analysis of spectral shape before, during, and after CT-DBS, respectively. Awake power spectra showed small local changes but few global changes over time (FIG. 3A). In the alpha range, FC6 initially demonstrated a spectral peak at ˜8-9 Hz which reduced in power but increased in frequency to ˜9-10 Hz by T4 (FIG. 3A, FC6 inset arrow). Following discontinuation of CT-DBS at T5, the FC6 power spectrum largely flattened and showed no clear peak within the alpha range (FIG. 3A, FC6 inset). A similar awake alpha modulation was present in C4 with a less prominent increase in alpha frequency from ˜8 Hz at T1 to ˜9 Hz at T4 (FIG. 3A, C4 inset arrow) and a complete flattening at T5 (FIG. 3A, C4 inset). Additional examination of parietal and occipital channels during wakeful periods also revealed increases in alpha frequency at T4 with slight reductions at T5. Channel C3 uniquely showed prominent electrophysiological change after CT-DBS was discontinued with the emergence of a clear spectral peak in the beta frequency range at ˜12 Hz during T5 (FIG. 3A, C3, asterisk).

In contrast to the variable results observed in the patient subject's awake EEG, spectral analysis of stage two sleep showed robust global changes over time. Power spectra were characterized by an increased peak frequency in the sleep spindle range from T1 to T4 across all channels (FIG. 3B). Following CT-DBS discontinuation at T5, power in the spindle range disappeared entirely in all channels except C3. At T5, C3 showed a continued increase in peak spindle frequency, albeit displaying a smaller and less defined spectral peak (FIG. 3B, C3 inset arrow). These findings are consistent with the observation of sleep spindle fragmentation across the majority of EEG channels following CT-DBS discontinuation.

SWS power spectra also demonstrated global changes over time, most notably characterized by an intrusion of 8-14 Hz power across channels prior to CT-DBS treatment at T1, corresponding to the presence of alpha-delta sleep (FIG. 3C). The 8-14 Hz alpha-delta sleep frequency signature was absent in all channels during active CT-DBS treatment at T4, only to re-emerge following CT-DBS discontinuation at T5. Re-emergence of alpha-delta sleep at T5 showed increased peak frequency in the alpha range across all except for the two frontal channels (F3 and F4).

### Relationship Between Sleep Dynamics and CT-DBS

For statistical comparison, data were collapsed into three groups: “Pre CT-DBS” (T1), “Active CT-DBS” (T2-T4), and “Post CT-DBS” (T5). Global feature measurements were compared across and within CT-DBS conditions for a quantitative analysis of the effects of CT-DBS treatment and subsequent cessation on EEG sleep dynamics.

### Stage Two Sleep Spindles

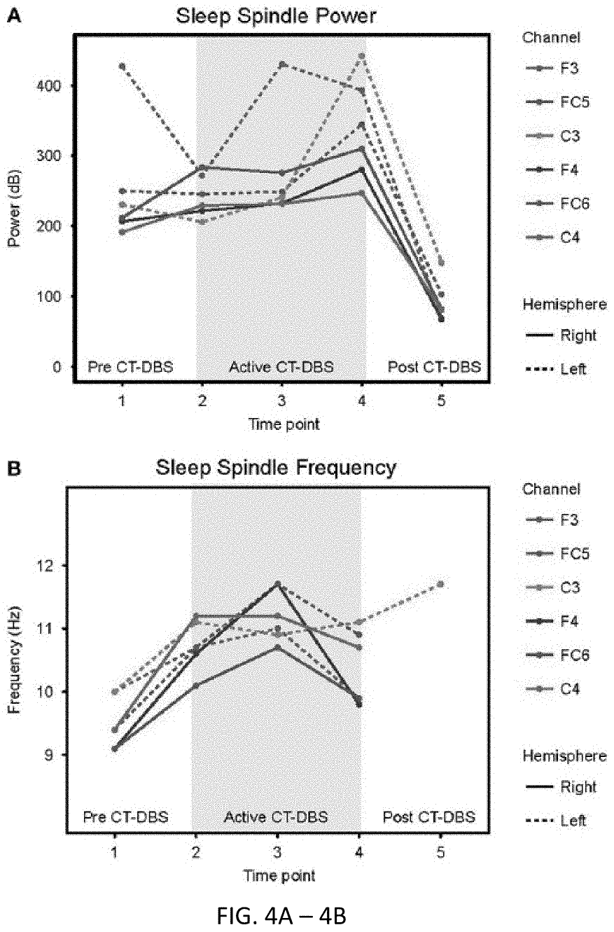

Normalized stage two power spectral calculations were used to quantify changes in spindle (9-16 Hz) power over time. A two-way ANOVA with factors CT-DBS (pre, active, post) and hemisphere (left, right) showed greater spindle power in the left hemisphere [F(1)=6.483, p=0.0177], as well as a highly significant main effect of CT-DBS condition [F(2)=20.411, p<0.0001]. Post-hoc tests identified a reduction in spindle power post CT-DBS compared to both pre and active CT-DBS conditions, p<0.001 and p<0.0001, respectively (FIG. 4A, Table 1). Spindle power remained consistent across the active CT-DBS condition, with the exception of a slight increase in the left hemisphere at T4 compared to T2, p=0.0438.

To quantify changes in spindle frequency, we first removed the post CT-DBS condition (T5) from analyses due to lack of spectral peak in the spindle range in five of the six channels (see FIG. 3B). A two-way ANOVA with factors CT-DBS (pre, active) and hemisphere (left, right) demonstrated significantly faster spindle frequency in both hemispheres during active CT-DBS than before CT-DBS treatment [F(1)=26.920, p<0.0001] (FIG. 4B, Table 1). Spindle frequency varied within active CT-DBS time points [F(2)=4.158, p=0.0425], such that there was a significant slowing from T3 to T4, p=0.0347, with frequency at T4 consistent with peak spindle frequency at T2.

TABLE 1
Average sleep variables calculated from EEG
power spectral estimates

| | | | Stage Two | | |
|---|---|---|---|---|---|
| | | Stage Two | Spindle | SWS | SWS |
| | CT-DBS | Spindle | Frequency | Delta | Alpha |
| | Condition | Power (dB) | (Hz) | Power (dB) | Power (dB) |
| Left | Pre | 302.62 ± | 9.80 ± | 23.26 ± | 232.04 ± |
| Hemi- | | 108.32 | 0.35 | 2.08 | 17.08 |
| sphere | Active | 313.61 ± | 10.89 ± | 104.22 ± | 100.82 ± |
| | | 90.04 | 0.48 | 83.29 | 18.17 |
| | Post | 106.76 ± | 11.70* | 45.30 ± | 293.66 ± |
| | | 39.86 | | 23.35 | 45.37 |
| Right | Pre | 203.35 ± | 9.20 ± | 27.30 ± | 184.90 ± |
| Hemi- | | 10.64 | 0.17 | 3.75 | 7.74 |
| sphere | Active | 256.80 ± | 10.66 ± | 91.39 ± | 98.10 ± 2 |
| | | 31.20 | 0.64 | 63.70 | 8.03 |
| | Post | 76.91 ± | NA | 46.10 ± | 216.06 ± |
| | | 8.20 | | 23.35 | 25.62 |

*Channel C3 only

### SWS Delta Power

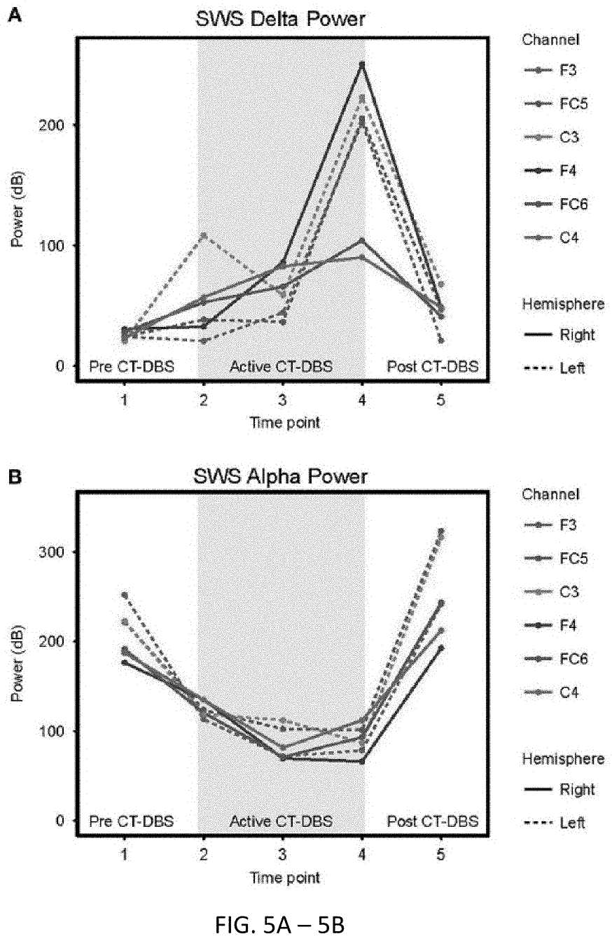

Delta (0.5-4 Hz) power was quantified from normalized SWS power spectra as an indicator of healthy SWS electrophysiology. A two-way analysis of variance with factors CT-DBS (pre, active, post) and hemisphere (left, right) showed a global effect of CT-DBS [F(2)=3.932, p=0.033] but not hemisphere, such that SWS delta power significantly increased from pre to active CT-DBS conditions, p=0.0472 (FIG. 5A, Table 1). During active CT-DBS, delta power was significantly greater at T4 than T2 and T3, p<0.001 and p=0.001, respectively. Despite an empirical reduction in delta power from T4 to T5, statistical analyses yielded no difference between active and post CT-DBS conditions, p=0.186.

### SWS Alpha Power

Alpha (8-14 Hz) power during SWS was quantified as a marker of alpha-delta sleep expression. A two-way ANOVA with factors CT-DBS (pre, active, post) and hemisphere (left, right) yielded a highly significant CT-DBS by hemisphere interaction [F(2)=5.657, p=0.00971]. Post hoc testing showed that global alpha power was reduced from pre to active CT-DBS conditions, p<0.001, followed by a significant rebound from active to post CT-DBS conditions, p<0.001. This rebound was larger in the left than the right hemisphere, such that SWS alpha power did not differ between hemispheres during pre or active CT-DBS conditions, but was greater in the left hemisphere following CT-DBS withdrawal, p=0.010 (FIG. 5B, Table 1). SWS alpha power also differed within active CT-DBS time points [F(2)=13.329, p<0.001], such that it was significantly reduced from T2 to T3, p=0.001, and remained suppressed at T4 compared to T2, p=0.003.

### III. Discussion

In this longitudinal study of a single patient subject in chronic minimally conscious state, the inventors report marked regression of sleep dynamics following the discontinuation of CT-DBS. In this patient subject, Adams et al. (Clin Neurophysiol (2016) 127:3086-3092) previously demonstrated that daytime CT-DBS (6 AM-6 PM) over a five year period was associated with the normalization of sleep architecture and dynamics; specific changes included increased spindle frequency during stage two sleep, increased sustained SWS, and the re-emergence of REM sleep. Presently, the inventors show regression of each of these improvements observed at one year after CT-DBS discontinuation (see FIG. 1B for a schematic summary) and in temporal correlation with a significant reduction in behavioral responsiveness (FIG. 2). During stage two sleep the inventors identify a loss of sleep spindles alongside a reduction in spectral power in the spindle range (FIGS. 3B, 4A-4B). The inventors also find a reversion of SWS delta power to pre-CT-DBS levels (FIGS. 3C & 5A), and no instances of REM sleep. Importantly, the inventors observe the re-emergence of a SWS-like frequency signature that had previously been suppressed by daytime CT-DBS (FIG. 5B). This frequency signature closely resembles the ‘alpha-delta sleep’ pattern that has been identified across several neuropsychiatric conditions, leading us to re-characterize this phenomenon as alpha-delta sleep arising within the severely injured brain. In summary, reduced behavioral responsiveness after CT-DBS discontinuation was associated with the abolishment of stage two sleep spindles, marked downregulation of SWS delta power, and the return of alpha-delta sleep. In the following sections the inventors discuss: 1) the proposed mechanism of sleep modulation by daytime CT-DBS in the severely injured brain and implications for sleep dynamics as an indicator of wakeful engagement, 2) altered network dynamics that may underlie alpha-delta sleep expression in the severely injured brain, and 3) a novel mechanistic framework for alpha-delta sleep generation across pathophysiologies.

### 1. Restoration of Frontostriatal Activation by CT-DBS May Drive Sleep Changes in the Severely Injured Brain

The rationale for using CT-DBS in minimally conscious state patients is two-fold: 1) the central thalamus has widespread innervation of frontal cortical and basal ganglia regions and plays a crucial role in maintaining arousal regulation during wakeful states, 2) multi-focal deafferentation is a characteristic injury pattern in severe brain injuries and is known to functionally and structurally disfacilitate central thalamic neuronal populations. The upregulation of central thalamic neurons via CT-DBS is therefore expected to re-establish frontostriatal neuronal firing rates, thereby restoring the frontocortical regulation of sustained waking arousal needed to support organized behavior. Studies of CT-DBS in another post-traumatic minimally conscious state patient provided proof-of-concept that restoration of sustained frontocortical activity correlates with improvements in organized behavior. Increased neuronal activity in the frontal cortices produced by CT-DBS was associated with heightened arousal, recovery of speech, restored executive motor control over one limb, and improved feeding behaviors. While CT-DBS in our patient subject failed to produce clinically measurable behavioral improvement, it did produce robust improvements in sleep electrophysiology. The inventors proposed that these sustained changes in network dynamics visible during sleep were the result of daytime activation of frontostriatal systems by CT-DBS, which allowed for organized neuronal activity in intact but functionally downregulated frontostriatal networks.

Here, the inventors show that CT-DBS cessation temporally correlated with significant regressions in sleep electrophysiology and behavioral responsiveness, providing strong support for the hypothesis that CT-DBS modulates sleep electrophysiology via upregulation of daytime frontostriatal activation and systems-level engagement. Modulation of sleep dynamics in response to diurnal neuronal activity has been well described in both animal and human studies. Across species, progressive wakefulness is associated with increased cortical excitability, increased neuronal firing rates, and increased extrasynaptic glutamate levels. Subsequent non-REM sleep episodes are characterized by an initial maintenance of high neuronal firing rates, increased cortical synchrony, and upregulated slow wave activity in regions corresponding to increased neuronal activation during wakefulness. Successive non-REM sleep episodes show a progressive decline in each of these features. Accounting for these sleep-wake dynamics, growing evidence indicates that wakefulness creates a net increase in synaptic strengths that requires sleep processes for renormalization; a concept known as the “synaptic homeostasis hypothesis” (SHY). Importantly, sustained high firing rates alone, such as those produced by CT-DBS, are insufficient to produce the changes in SWS observed here. Rather, SWS changes are more likely to result from system-level wakeful engagement with the environment that results in synaptic potentiation. The SHY therefore predicts that the marked improvements in sleep electrophysiology seen in our patient subject during CT-DBS reflect fundamental changes in synaptic potentiation occurring during wakefulness. Further supporting this inference, daytime CT-DBS is known to upregulate the long-term potentiation (LTP)-related immediate early gene zif268 within neocortex. Similar gene expression patterns are expressed during periods of REM sleep following wakeful LTP; rodent studies have implicated these REM periods as instrumental in the consolidation of CT-DBS-induced learning. Accordingly, the selective appearance of REM sleep with CT-DBS in our patient subject likely reflects changes in LTP-related gene expression induced by the daily 12-hour CT-DBS periods. Taken together, the inventors' findings suggest that the restoration of both non-REM sleep architecture and REM sleep episodes during CT-DBS may provide an indirect marker of meaningful daytime engagement across a range of sensory and associative processing systems within the forebrain.

The finding that improvements in sleep electrophysiology are lost following withdrawal of CT-DBS suggests further that this process can be reversed. Specifically, sub-threshold wakeful activation may insufficiently engage organized neuronal dynamics needed for synaptic potentiation. Under-activated networks would therefore fail to produce the homeostatic sleep pressure necessary for large-scale neuronal synchronization and synaptic scaling during sleep. This general mechanism has precedence in the healthy brain. Following periods of arm immobilization, healthy individuals demonstrate localized wakeful synaptic depression and reductions in sleep slow wave activity over contralateral sensorimotor cortex. In the deafferented brain, the reduction of thalamocortical outflow associated with CT-DBS discontinuation would be expected to result in decreased cortical activation and synaptic depression, culminating in a progressive loss of wakeful frontocortical excitability and diminished homeostatic sleep-wake processes over time. The reduced behavioral responsiveness and degradation of organized sleep architecture after CT-DBS withdrawal at T5 supports this inference. Collectively, these observations raise the possibility that restoration of synaptic homeostasis during sleep may be a process that is re-engaged in the severely injured brain only after sufficient increases in large-scale organized neuronal firing patterns emerge across the cerebral cortex to produce a net increase in synaptic strength during wakefulness. Such reinstatement of large-scale network engagement, including both glutamatergic synaptic potentiation and GABAergic firing rates, provides a testable mechanism for the observed changes in sleep architecture with CT-DBS.

As an exception to the observed global regressions following CT-DBS discontinuation, channel C3 displayed retained sleep spindles and improvements in SWS delta power at T5 (FIGS. 3B-3C), as well as the emergence of high frequency beta and healthy ‘Mu’ or ‘wicket’ rhythms (˜8-13 Hz) during wake (57,58) (FIG. 3A). Of note, although this patient subject was unable to communicate, he retained a high-level of emotional responsiveness consistent with his sense of humor prior to the injury. These unique dynamics underscore the structural preservation of the patient subject's left temporal cortex as well as verify the functional preservation of his left temporal language processing capabilities. Mechanistically, continued improvements in cortical regions underlying C3 may have resulted from local restructuring as the result of restored neuronal activation across relatively preserved cortical substrate during CT-DBS. Such changes would not be unprecedented; Thengone and colleagues (Sci Transl Med (2016) 8:368re5-368re5) recently demonstrated prominent changes in structural connectivity emanating from Broca's area following implementation of assistive communication technology in a minimally conscious state patient. This independent EEG pattern exhibited by a localized brain region in our patient subject underscores the impact that upregulated neuronal activation can have on the recovery of functional circuitry in structurally intact brain regions.

### 2. An Underactive Pre Frontal Cortex May Permit Ventral Limbic Over-Activation, Resulting in Alpha-Delta Sleep

Perhaps the most novel and interesting finding observed here is the mixing of alpha and delta rhythms during sleep, originally reported by Adams et al. (2016) and identified here as alpha-delta sleep. Although the functional role and underlying circuit mechanisms of alpha-delta sleep have remained elusive, the phenomenon has been reported in a variety of conditions including fibromyalgia/chronic fatigue, rheumatoid arthritis, schizophrenia, major depressive disorder with implications for suicidality, anxiety, and in healthy individuals with induced pain and/or arousal during sleep. To the best of the inventors' knowledge, this is the first report of alpha-delta sleep in the severely injured brain. The persistence of the alpha-delta sleep phenotype across a range of neurological conditions, and now severe brain injury, invites us to consider a common underlying mechanism.

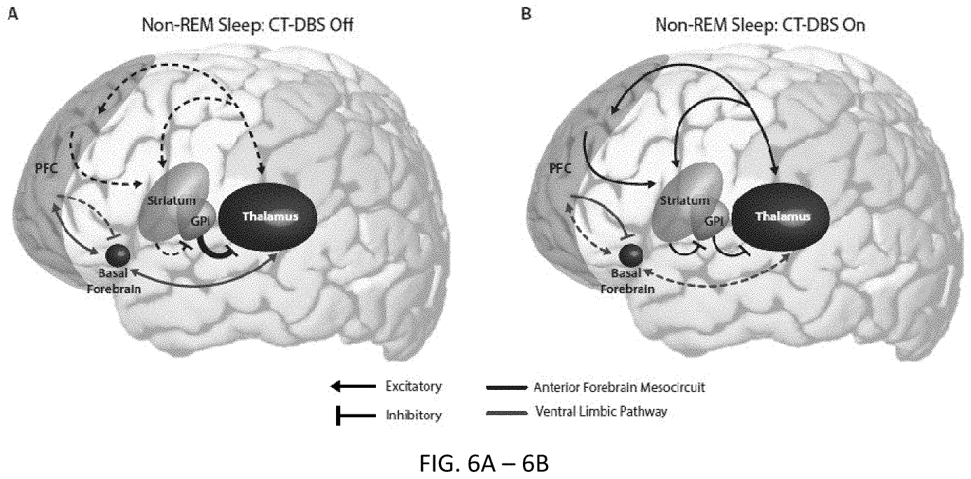

The inventors observe that the conditions in which alpha-delta sleep is reported fall into two mechanistic categories: 1) those characterized by a primary pathology of cerebral hypofrontality or 2) those characterized by a primary upregulation of ventral limbic activation. Both mechanisms result in an increase in limbic system activity during sleep, either via under-activation of the descending corticothalamic pathway needed to drive homeostatic sleep pressure or an overactivation of the ascending pathways that maintain wakefulness. Accordingly, the inventors hypothesize that the appearance of alpha-delta sleep is indicative of a failure of the prefrontal cortex to sufficiently inhibit excitatory output from ventral structures to the thalamus during the shift into synchronized cortical activity for SWS (FIG. 6). Specifically, the inventors identify the basal forebrain as a likely generator of thalamic depolarization in alpha-delta sleep due to its cholinergic projections to the thalamus. Support for this mechanism is provided by simulation and in vitro studies of both alpha oscillations and alpha-delta sleep expression. Regarding alpha production by basal forebrain cholinergic projections, the activation of muscarinic acetylcholine receptors on reticular nuclei, thalamocortical, and high-threshold thalamocortical cells, as well as on somatosensory and visual thalamic nuclei, has been evidenced to produce alpha oscillations in thalamic models and cat in vitro slice recordings, respectively. Conversely, follow-up in vitro studies show that direct thalamic application of a muscarinic acetylcholine receptor antagonist reduces high-threshold thalamocortical cell bursting, and in turn thalamic and cortical alpha power. Additional simulation studies demonstrate that alpha-delta sleep generation may originate in aberrant thalamic depolarization during SWS, specifically of ‘high-threshold’ thalamocortical cells that serve as the putative generators of awake alpha.

Critically, the novel finding that alpha-delta sleep is modulated by CT-DBS lends strong support to the validity of this prefrontal-ventral dysfunction model of alpha-delta sleep generation (see FIG. 6 schematic). In the patient subject, CT-DBS restored bulk activation of the frontal cortices during the day, likely facilitating the reinstatement of top-down limbic inhibition and driving activity-dependent increases in homeostatic sleep pressure. This increase in frontocortical GABAergic tone would be expected to carry over into sleep via sustained alterations of GABAergic firing rates and the mutual reinstatement of synchronous cortical slow wave activity needed for synaptic scaling (see Allada, Cirelli, & Sehgal, 2017 for a detailed review). With proper inhibition of ventral limbic structures by the frontal cortex during SWS, there would be minimal excess corticothalamic excitation and therefore attenuation of the alpha-delta sleep phenotype (72). In our patient subject, when CT-DBS was eventually discontinued, daytime frontocortical network activation was reduced, likely resulting in a gradual lifting of frontal inhibition over limbic structures during SWS and the observed re-emergence of alpha-delta sleep.

### 3. Restoring Frontocortical GABAergic Tone Reduces Alpha-Delta Sleep

The prefrontal-ventral dysfunction model of alpha-delta sleep provides a consistent mechanism across several conditions in which alpha-delta sleep has been documented. Patients with fibromyalgia demonstrate reduced gray matter volume of the frontal cortex alongside increased structural connectivity of the amygdala. Schizophrenia is associated with prefrontal GABAergic deficits and thalamocortical hypoconnectivity. Major depression is characterized by prefrontal gray matter reductions and GABAergic interneuron deficits, as well as ventral hypermetabolism that persists from wakefulness into non-REM sleep. Individuals with anxiety and PTSD demonstrate reduced prefrontal regulation of the amygdala, which is present in both fear conditions and resting states. The high prevalence of sleep disturbances in PTSD suggests that prefrontal-amygdala dysfunction persists into sleep states as well. Together, these commonalities suggest that alpha-delta sleep dynamics may be indicative of the presence and/or severity of prefrontal-ventral dysregulation across behavioral diagnoses.

In support of the generalizability of this model, the alleviation of frontal GABAergic deficits by gamma hydroxybutyrate (GHB/sodium oxybate) has been found to suppress alpha-delta sleep in several conditions. GHB is an activity-dependent neurotransmitter synthesized within GABAergic interneurons. Importantly, these GHB-containing interneurons play a critical role in the endogenous regulation of sleep-wake cycles by inhibiting cholinergic structures such as the basal forebrain. At exogenous doses, GHB exerts inhibitory effects by directly binding GABA-B receptors; a necessary step for the homeostatic modulation of firing rates. Accordingly, the demonstrated suppression of alpha-delta sleep and dose-dependent upregulation of SWS by GHB likely occurs through a GABA-B receptor-mediated process analogous to both the endogenous production of homeostatic sleep pressure through normal wakeful activity and the exogenous driving of organized frontocortical networks with CT-DBS. The inventors suggest that these findings underscore the bi-directional carry-over effects of GABAergic upregulation between sleep and wakeful states. In the patient subject, upregulation of frontocortical GABAergic circuits during wakefulness resulted in the recovery of sleep architecture. Accordingly, the inventors emphasize the notion that state-dependent activation of GABAergic circuits exerts a 24-hour cyclical influence over organized neuronal function.

### Example 2. Effects of Sodium Oxybate on Secondary Blast Injury Brain Damage

The inventors inflicted a secondary-blast injury (SBI) to mice targeting the anterior forebrain mesocircuit, which corresponds to the frontal/prefrontalcortical-pallidal thalamocortical loop systems.

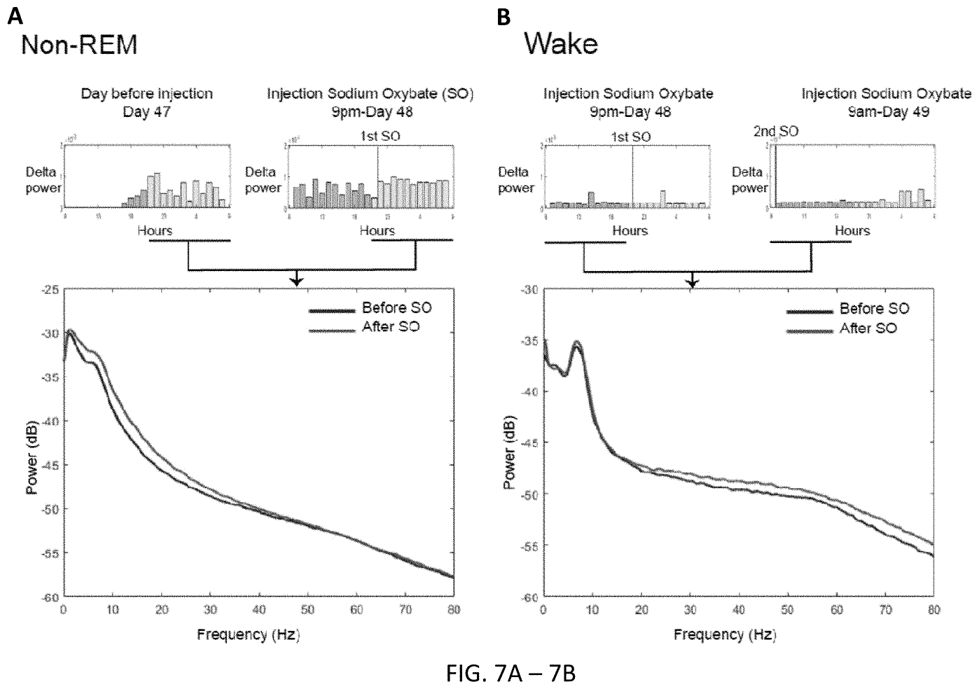

The inventors examined animals' sleep-wake cycle before, after the SBI, and while injecting sodium oxybate (400 mg/Kg; FIG. 7A-7B). Local field potentials (LFPs) were recorded from the frontal area and neck-EMG activity to detect and extract the different sleep/wake cycle components—Non-REM, REM, and Wake state. The inventors examined the whole range of biological LFP frequencies before and after sodium oxybate injection by calculating power spectral density using Thomson's multi-taper method implemented using the Chronux tool box from MATLAB.

The inventors mainly observed an increase in the delta oscillations (1-4 Hz) in the Non-REM when the drug was injected right before the sleep time (9 pm), remaining stable for hours. Before the injury, the delta power remains stable during Non-REM, suggesting that sodium oxybate restores the steady-state of delta power. In contrast, the inventors observed higher beta (20-40 Hz) and gamma (>40 Hz) frequencies during the wake state post-sodium oxybate injection when they calculated the wake power spectra, suggesting a more aroused cortical state.

An increase in beta and gamma frequency power is associated with the recovery of complex behaviors in a wakeful rodent state and in humans. Thus, application of sodium oxybate during the night results in elevation of delta (1-4 Hz) LFP power and increases during the subsequent wakeful period in beta (20-40 Hz) and gamma (>40 Hz) LFP. Importantly, this is consistent with our theory because of the short half-life of sodium oxybate: these changes in wakeful LFP power reflect alteration of cortical neuronal properties linked to the increased delta during sleep induced by sodium oxybate. Similarly, the elevation of delta power does not persist during the wakeful period.

1. A method for improving a wakeful function in a subject suffering from an acquired cognitive deficiency, comprising administering to the subject an effective amount of an upregulator of GABA_{B} signaling, thereby improving the wakeful function in the subject.

2. The method of claim 1, wherein the administering step is performed at night.

3. The method of claim 1, wherein the upregulator of GABA_{B} signaling is selected from the group consisting of baclofen, tiagabine, lesogaberan (AZD3355), GS-39783, zolpidem, midazolam, and sodium oxybate.

4. The method of claim 1, wherein the upregulator of GABA_{B} signaling is represented by the chemical formula:

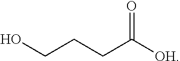

5. The method of claim 1, wherein the upregulator of GABA_{B} signaling is sodium oxybate with the following chemical formula:

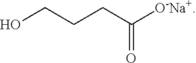

6. The method of claim 1, wherein the upregulator of GABA_{B} signaling is represented by the chemical formula: 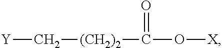
wherein X is H, a pharmaceutically acceptable cation or (C₁-C₄)alkyl, and Y is OH, (C₁-C₄)alkoxy, (C₁-C₄) alkanoyloxy or benzoyloxy.

7. The method of claim 6, wherein Y is OH or (C₁-C₄) alkanoyloxy.

8. The method of claim 6, wherein X is Na⁺.

9. The method of claim 1, wherein the upregulator of GABA_{B} signaling comprises electrical stimulation of corticothalamic afferents in the subject.

10. The method according to claim 1, wherein the acquired cognitive deficiency results from an insult selected from the group consisting of stroke, toxicological agents, anoxia, ischemia, nutritional deficiencies, developmental diseases, infectious diseases, neoplastic diseases, and degenerative diseases.

11. The method according to claim 1, wherein the acquired cognitive deficiency results from a traumatic brain injury.

12. The method according to claim 1, wherein the wakeful function is selected from the group consisting of perceptual awareness, memory, intellect, learning ability, logic ability, attention and executive function.

13. The method according to claim 1, wherein the method also improves one or more features of sleep selected from the group consisting of depth of slow wave modulation, frequency of spindles, REM sleep, circadian rhythmicity, and sleep architecture.

14. A method for improving a wakeful function in a subject suffering from an acquired cognitive deficiency, comprising administering to the subject an effective amount of an upregulator of GABA_{B} signaling at night, in combination with administering an anterior-forebrain stimulating therapy when the subject is awake, thereby improving the wakeful function in the subject.

15. The method of claim 14, wherein the anterior-forebrain stimulating therapy comprises administering a small molecule compound selected from the group consisting of amantadine hydrochloride, methylphenidate, donazepil, noradrenaline, and ketamine.

16. The method of claim 14, wherein the anterior-forebrain stimulating therapy is selected from the group consisting of deep brain stimulation, transcranial direct current stimulation, optogenetic stimulation, and focused ultrasound.

17. The method according to claim 14, wherein the upregulator of GABA_{B} signaling is selected from the group consisting of baclofen, tiagabine, lesogaberan (AZD3355), GS-39783, zolpidem, midazolam, and sodium oxybate.

18. The method according to claim 14, wherein the upregulator of GABA_{B} signaling is represented by the chemical formula:

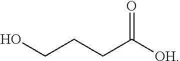

19. The method according to claim 14, wherein the upregulator of GABA_{B} signaling is sodium oxybate represented by the chemical formula:

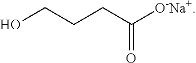

20. The method according to claim 14, wherein the upregulator of GABA_{B} signaling is represented by the chemical formula 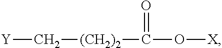
wherein X is H, a pharmaceutically acceptable cation or (C₁-C₄)alkyl, and Y is OH, (C₁-C₄)alkoxy, (C₁-C₄) alkanoyloxy or benzoyloxy.

21. The method of claim 20, wherein Y is OH or (C₁-C₄) alkanoyloxy.

22. The method of claim 20, wherein X is Na⁺.

23. The method according to claim 1, wherein the upregulator of GABA_{B} signaling comprises electrical stimulation of corticothalamic/thalamocortical afferents in the subject.

24. The method according to claim 14, wherein the acquired cognitive deficiency results from an insult selected from the group consisting of stroke, toxicological agents, anoxia, ischemia, nutritional deficiencies, developmental diseases, infectious diseases, neoplastic diseases, and degenerative diseases.

25. The method according to claim 14, wherein the acquired cognitive deficiency results from a traumatic brain injury.

26. The method according to claim 14, wherein the wakeful function is selected from the group consisting of perceptual awareness, memory, intellect, learning ability, logic ability, attention and executive function.

27. The method according to claim 14 14-26, wherein the method also improves one or more features of sleep selected from the group consisting of depth of slow wave modulation, frequency of spindles, REM sleep, circadian rhythmicity, and sleep architecture.
